(19) United States
(12) Patent Application Publication
(10) Pub. No.: US 2024/0165002 A1
(43) Pub. Date: May 23, 2024

(54) Compositions Incorporating Avobenzone Complexes

(71) Applicant: University of Cincinnati, Cincinnati, OH (US)

(72) Inventors: Harshita Kumari, Mason, OH (US); Jinnipha Pajoubpong, Cincinnati, OH (US); Xu Kang, Cincinnati, OH (US)

(73) Assignee: University of Cincinnati, Cincinnati, OH (US)

(21) Appl. No.: 18/517,667

(22) Filed: Nov. 22, 2023

Related U.S. Application Data
(60) Provisional application No. 63/427,425, filed on Nov. 22, 2022.

(51) Int. Cl. A61K 8/42; A61K 8/35; A61Q 17/04

(52) U.S. Cl. CPC A61K 8/42; A61K 8/35; A61Q 17/04

(57) ABSTRACT

Avobenzone complexes are provided, including a complex of avobenzone-RsC5 and a complex of cucurbit[7]uril. Among other uses, the complexes can be used in sunscreen compositions. In one embodiment, the avobenzone complex is from about 0.5 to about 10 weight percent of the sunscreen composition.

### CROSS REFERENCE TO RELATED APPLICATIONS

This application claims priority to U.S. Provisional Application Ser. No. 63/427,425, filed Nov. 22, 2022, which application is hereby incorporated by reference in its entirety.

### TECHNICAL FIELD

The present invention relates to complexes of avobenzone.

### BACKGROUND OF THE INVENTION

There are handful of UVA filters and absorbers approved by US-FDA, including avobenzone (AVB). Most broad-spectrum sunscreen products in the market contain avobenzone because of its effectiveness. However, its UVA absorbance performance can be compromised due to the lack of photostability. Therefore, a need still exists to improve the photostability of avobenzone.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention.

In an embodiment of the invention, a composition comprising the complex avobenzone-RsC5 is provided. In one embodiment, a sunscreen composition is provided that includes the avobenzone-RsC5 complex and a cosmetically acceptable carrier. In another embodiment, the avobenzone-RsC5 complex is from about 0.5 to about 10 weight percent of the sunscreen composition.

In one embodiment, the sunscreen composition also includes one or more film formers. In another embodiment, the sunscreen composition also includes one or more emulsifiers. In one embodiment, the sunscreen composition also includes one or more silicone oils. In another embodiment, the sunscreen composition is in the form of a lotion, cream, gel, or spray. In one embodiment, the cosmetically acceptable carrier in the sunscreen composition is water.

In another embodiment of the invention, a composition comprising the complex avobenzone-cucurbit[7]uril is provided. In one embodiment, a sunscreen composition is provided that includes the avobenzone-cucurbit[7]uril complex and a cosmetically acceptable carrier. In another embodiment, the avobenzone-cucurbit[7]uril complex is from about 0.5 to about 10 weight percent of the sunscreen composition.

In one embodiment, the sunscreen composition also includes one or more film formers. In another embodiment, the sunscreen composition also includes one or more emulsifiers. In one embodiment, the sunscreen composition also includes one or more silicone oils. In another embodiment, the sunscreen composition is in the form of a lotion, cream, gel, or spray. In one embodiment, the cosmetically acceptable carrier in the sunscreen composition is water.

### BRIEF DESCRIPTION OF THE DRAWINGS

FIG. 1 is an illustration of the chemical structure of avobenzone.

FIG. 2 is an illustration of the chemical structure of the calix[n]resorcinarene RsC5.

FIG. 3 is a graph showing NMR titration spectra of AVB+RsC5 at different mol ratios in acetonitrile-d3.

FIG. 4 is a graph showing f NMR spectra of AVB+RsC1 at mol ratio of 1:1 in acetonitrile-d3.

FIG. 5 is a schematic showing the potential complexing of avobenzone with cucurbit[7]uril (CB7).

FIG. 6 is a schematic showing the complex of avobenzone with cucurbit[7]uril (CB7) to improve photostability and reduce skin permeation.

FIG. 7 is a synthesis scheme for cucurbit[n]uril.

FIG. 8 is a schematic showing a method for isolating CB7.

FIG. 9 is a schematic showing a preparation method using physical mixtures.

FIG. 10 is a graph showing that CB7 shifts the tautomeric equilibrium of avobenzone.

FIG. 11 is a graph showing Differential Scanning Calorimetric (DSC) Analysis of avobenzone, CB7 and various mixtures.

FIG. 12 is a graph showing an ATR-FTIR Spectrum analysis of co-ground Avobenzone+CB7 (1:1) with H₂O VS CB7.

FIG. 13 is a graph showing UV-vis spectra of samples prepared for Job's plot.

FIG. 14 is a graph showing a Job's plot for avobenzone and CB7 by plotting the difference in Area under Curve (AUC) ranging from 220 to 450 nm versus the molar ratio of CB7.

FIG. 15A is a graph showing the UV absorbance of AVB alone in 10% ethanol/water before and after 60-min irradiation.

FIG. 15B is a graph showing the UV absorbance of AVB-CB[7] (1:5 molar ratio) in 10% ethanol/water before and after 60-min irradiation

FIG. 15C is a graph showing the UV absorbance of AVB-CB[7] (1:10 molar ratio) in 10% ethanol/water before and after 60-min irradiation

FIG. 16 is a graph showing the results of the UV absorbance data expressed as % recovery.

### DETAILED DESCRIPTION OF THE INVENTION

The details of one or more embodiments of the disclosed subject matter are set forth in this document. Modifications to embodiments described in this document, and other embodiments, will be evident to those of ordinary skill in the art after a study of the information provided herein.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

The compositions and methods of the present disclosure can comprise, consist of, or consist essentially of the essential elements and limitations of the disclosure described herein, as well as any additional or optional ingredients, components, or limitations described herein or otherwise useful.

All percentages, parts and ratios herein are based upon the total weight of the compositions of the present disclosure, unless otherwise indicated.

As used herein, the term “about,” when referring to a value or to an amount of mass, weight, time, volume, pH, size, concentration or percentage is meant to encompass variations of in some embodiments±20%, in some embodiments±10%, in some embodiments±5%, in some embodiments±1%, in some embodiments±0.5%, and in some embodiments±0.1% from the specified amount, as such variations are appropriate to perform the disclosed method.

“Cosmetically acceptable” means that the item in question is compatible with a keratinous substrate such as skin and hair. For example, a “cosmetically acceptable carrier” means a carrier that is compatible with a keratinous substrate such as skin and hair.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

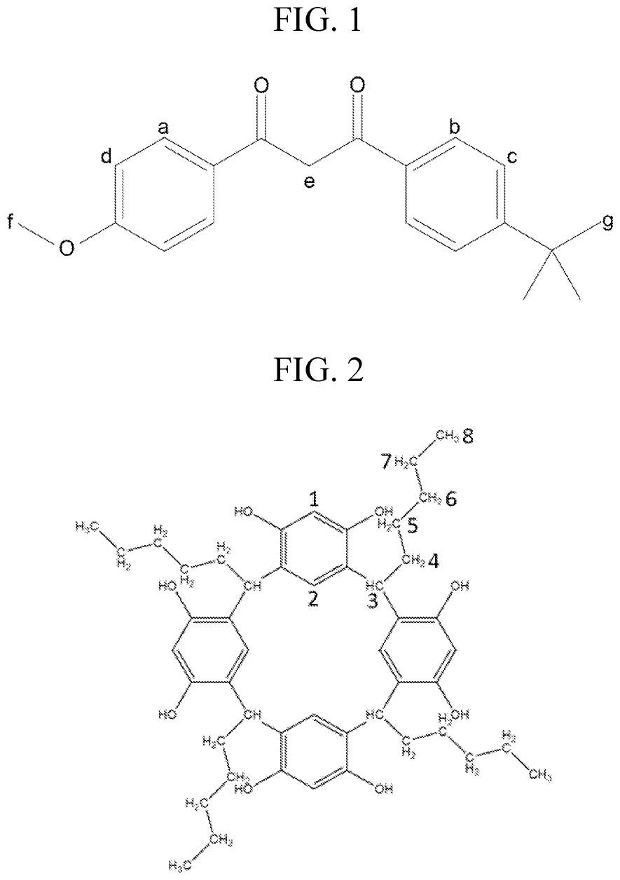

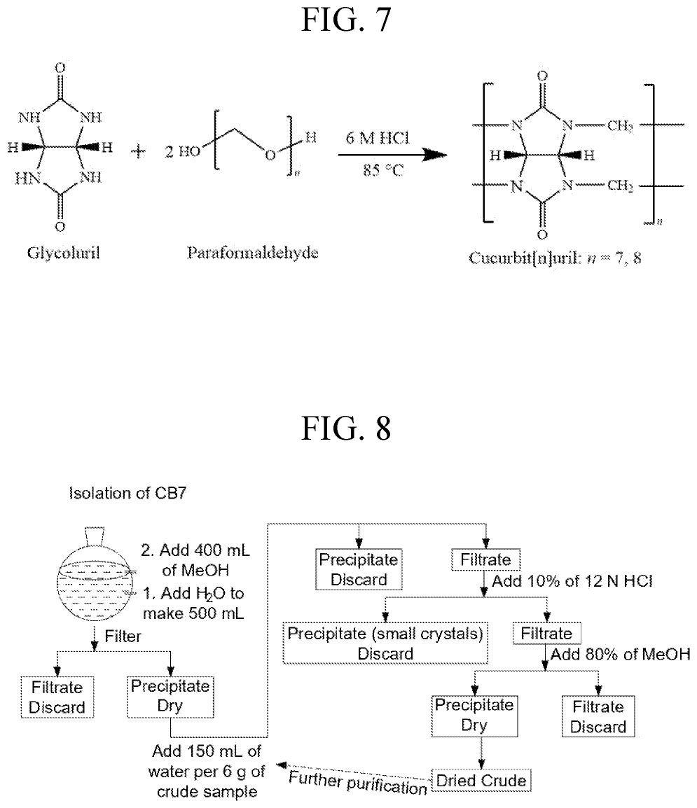

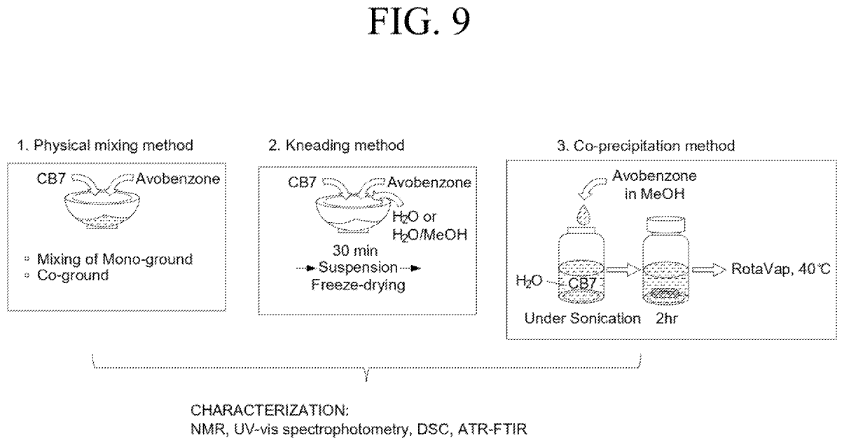

The photo-stability of sunscreen actives, the toxicological impacts of photo-degradation products and controlling skin penetration are three of the major challenges for current sunscreen products. Sunscreens such as oxybenzone/benzophenone-3 are emerging as environmental contaminants, impacting coral reefs. Some sunscreens have contaminated humans as well. They have been detected in human urine in 97% of the population. Thus, skin penetration across deeper layers (dermis) is a major concern with organic photoactives. Although macromolecular quantitative estimation of skin penetration of sunscreen is possible via radiolabelled ¹⁴C tape stripping assays on human/pig skin, a nanometric molecular level understanding is still lacking. The present invention involves an organic active (avobenzone, FIG. 1) complexed with either RsC5 (FIG. 2) or cucurbit[7]uril (CB7) (FIG. 7).

In one embodiment of the present invention, a calix[n]resorcinarene (RsC5, FIG. 2) is used as the host molecule complexed with avobenzone (FIG. 1). In another embodiment of the present invention, cucurbit[7]uril (CB7) is used as the host molecule complexed with avobenzone. Avobenzone (1-(4-methoxyphenyl)-3-(4-tertbutylphenyl)-propane-1,3-dione), also known as Parsol 1789, is a broad-spectrum sunscreen agent. Its ability to absorb both UVA and UVB radiations makes it one of the more useful sunscreen agents that prevents photodamage of skin. However, once exposed to sun, avobenzone offers only 30 minutes of photoprotection. Hence, it is formulated with photostabilizers, such as octocrylene, which is a known endocrine disrupter and releases free radicals. Experimental and theoretical studies suggest that the chelated enol form is the ground state. Upon UV irradiation in polar nonprotic solvents, the enol form converts to the keto tautomer. The present invention has found rather inert macrocycles that can provide a confined environment for photostability and/or controlled penetration across bilayer.

### Avobenzone

Avobenzone (AVB) is a dibenzoylmethane derivative that absorbs the full UVA spectrum (320-400 nm) and has a relatively high molar absorption coefficient (33,756 L/(mol·cm) at 360 nm), resulting in a high UV absorbance efficiency. Consequently, it is the most common active ingredient in chemical sunscreen formulations and has been approved for use globally (up to 3% in the USA and Canada; up to 5% in the EU, UK, and Australia; and up to 10% in Japan). However, AVB exhibits poor photostability because it undergoes keto-enol tautomerization upon exposure to light. The enol tautomer, which predominates in solution, absorbs UVA radiation and converts into the keto form. Notably, the keto form absorbs UVC instead of UVA radiation. The singlet-excited-state keto form may further convert into the triplet-excited-state keto form, which can either return to the ground state and eventually regenerate the original enol or react with nearby molecules to produce degradation products, resulting in a loss of UVA protection.

The keto-enol equilibrium of AVB plays a crucial role in its photostability upon complexation. In one embodiment, the present invention improves the photostability of AVB by complexing AVB with the macrocyclic host cucurbit[7]uril (CB[7]), which can shift the equilibrium toward a keto tautomeric form. Its cavity size (inner diameter of 7.3 Å[15]), makes CB[7] well suited for manipulating the equilibrium between the keto and enol forms of AVB.

### Cosmetically Acceptable Carrier

In one embodiment, the avobenzone complexes of the present invention are useful as an ingredient of sunscreen compositions. In one embodiment, the avobenzone complex comprises from about 0.5 to about 10 weight percent of the sunscreen composition. In another embodiment, the avobenzone complex comprises from about 1 to about 5 weight percent of the sunscreen composition. The sunscreen compositions include a cosmetically acceptable carrier. The phrase “cosmetically acceptable” means that the material is compatible with skin and hair. For example, “cosmetically acceptable carrier” means a carrier that is compatible with skin and hair, and is acceptable for application to the body.

The cosmetically acceptable carrier may include, for example, water and/or water soluble solvents. Non-limiting examples of cosmetically acceptable carriers include glycerin, C1-4 alcohols, organic solvents, fatty alcohols, fatty ethers, fatty esters, polyols, glycols, vegetable oils, mineral oils, liposomes, laminar lipid materials, water, or any combinations thereof.

As examples of organic solvents, non-limiting mentions can be made of monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds.

In some instances, cosmetically acceptable carriers may comprise water, a mixture of water and at least one cosmetically acceptable organic solvent, or at least one cosmetically acceptable organic solvent. Additionally, cosmetically acceptable carriers may be or may include ethanol, a glycol ether, for example, dipropylene glycol n-butyl ether, isododecane, mineral oil, propylene glycol, pentylene glycol, hexylene glycol, glycerol, and mixtures thereof.

The total amount of cosmetically acceptable carrier can vary, but in some instances may be about 50 to about 95 wt. %, based on the total weight of the sunscreen composition. In some instances, the total amount of cosmetically acceptable carrier is about 55 to about 95 wt. %, about 60 to about 95 wt. %, about 70 to about 95 wt. %, about 75 to about 95 wt. %, about 50 to about 90 wt. %, about 60 to about 90 wt. %, about 70 to about 90 wt. %, or about 75 to about 90 wt. %, based on the total weight of the sunscreen composition.

### Film Formers

Film-formers may be incorporated into the sunscreen compositions to ensure even coverage of UV filters and can be used to render the composition water resistant. The film former is typically a hydrophobic material that imparts film forming and/or waterproofing characteristics. One such agent is polyethylene, which is available from New Phase Technologies as Performalene® 400, a polyethylene having a molecular weight of 400. Another suitable film former is polyethylene 2000 (molecular weight of 2000), which is available from New Phase Technologies as Performalene®. Yet, another suitable film former is synthetic wax, also available from New Phase Technologies as Performa® V-825. Other typical film-formers include acrylates/acrylamide copolymer, acrylates copolymer, acrylates/C12-C22 alkylmethacrylate copolymer, polyethylene, waxes, VP/dimethiconylacrylate/polycarbamylpolyglycol ester, butylated PVP, PVP/hexadecene copolymer, octadecene/MA copolymer, PVP/eicosene copolymer, tricontanyl PVP, Brassica Campestris/Aleuritis Fordi Oil copolymer, decamethyl cyclopentasiloxane (and) trimethylsiloxysilicate, and mixtures thereof. In some cases, the film former is acrylates/C12-C22 alkylmethacrylate copolymer sold under the tradename Allianz OPT® by ISP.

The total amount of film forming in the sunscreen compositions, if present, may vary but is typically about 0.1 to about 40 wt. %, based on the total weight of the sunscreen composition. In some instances, the total amount of film formers may be about 0.1 to about 30 wt. %, about 0.1 to about 20 wt. %, about 0.1 to about 10 wt. %, about 1 to about 40 wt. %, about 1 to about 30 wt. %, about 1 to about 20 wt. %, or about 1 to about 10 wt. %, based on the total weight of the sunscreen composition.

### Emulsifiers

The sunscreen compositions may optionally include at least one emulsifier such as an amphoteric, anionic, cationic or nonionic emulsifier, used alone or as a mixture, and optionally a co-emulsifier. Emulsifiers are most often used when the sunscreen composition is in the form of an emulsion. The emulsifiers are chosen in an appropriate manner according to the emulsion to be obtained (W/O or O/W).

The total amount of emulsifiers in the sunscreen compositions, if present, may vary but are typically about 0.1 to about 30 wt. %, based on the total weight of the sunscreen composition. In some instances, the total amount of emulsifiers is about 0.1 to about 20 wt. %, about 0.1 to about 15 wt. %, about 0.1 to about 10 wt. %, about 0.5 to about 30 wt. %, about 0.5 to about 20 wt. %, about 0.5 to about 15 wt. %, about 0.5 to about 10 wt. %, about 1 to about 30 wt. %, about 1 to about 20 wt. %, about 1 to about 15 wt. %, about 1 to about 10 wt. %, or about 5 to about 5 wt. %, based on the total weight of the sunscreen composition.

### Silicone Oils

The sunscreen compositions may optionally include one or more silicone oils. Non-limiting examples of silicone oils include dimethicone, cyclomethicone, polysilicone-11, phenyl trimethicone, trimethylsilylamodimethicone, and stearoxytrimethylsilane. In some cases, the cosmetic composition includes dimethicone, and optionally additional oils, including additional silicone oils. Typically, the one or more silicone oils is a non-volatile silicon oil. In some embodiments, the silicone oil is polydimethylsiloxanes (PDMSs), polydimethylsiloxanes comprising alkyl or alkoxy groups which are pendent and/or at the end of the silicone chain, which groups each contain from 2 to 24 carbon atoms, or phenyl silicones, such as phenyl trimethicones, phenyl dimethicones, phenyl(trimethylsiloxy)diphenylsiloxanes, diphenyl dimethicones, diphenyl(methyldiphenyl)trisiloxanes or (2-phenylethyl)trimethylsiloxysilicates.

The total amount of silicone oils in the sunscreen compositions, if present, can vary but is typically about 0.1 to about 40 wt. %, based on the total weight of the sunscreen composition. In some cases, the total amount of silicone oils in the sunscreen composition may be about 0.1 to about 30 wt. %, about 0.1 to about 20 wt. %, about 0.1 to about 10 wt. %, about 1 to about 40 wt. %, about 1 to about 30 wt. %, about 1 to about 20 wt. %, about 1 to about 10 wt. %, about 5 to about 40 wt. %, about 5 to about 30 wt. %, about 5 to about 20 wt. %, or about 5 to about 15 wt. %, based on the total weight of the sunscreen composition.

### Forms

The form of the sunscreen compositions is not limited. For example, the sunscreen compositions may be in the form of a cream, a gel, a paste, a lotion, a rinse, a foam, an emulsion, a spray, etc. The sunscreen compositions may be prepared according to techniques that are well known to those skilled in the art, in particular those intended for the preparation of emulsions of oil-in-water or water-in-oil type. They may be in particular in the form of a simple or complex emulsion (O/W, W/O, O/W/O or W/O/W emulsion) such as a cream or a milk, in the form of a gel or a cream-gel, or in the form of a lotion.

### EXAMPLES

### Example 1

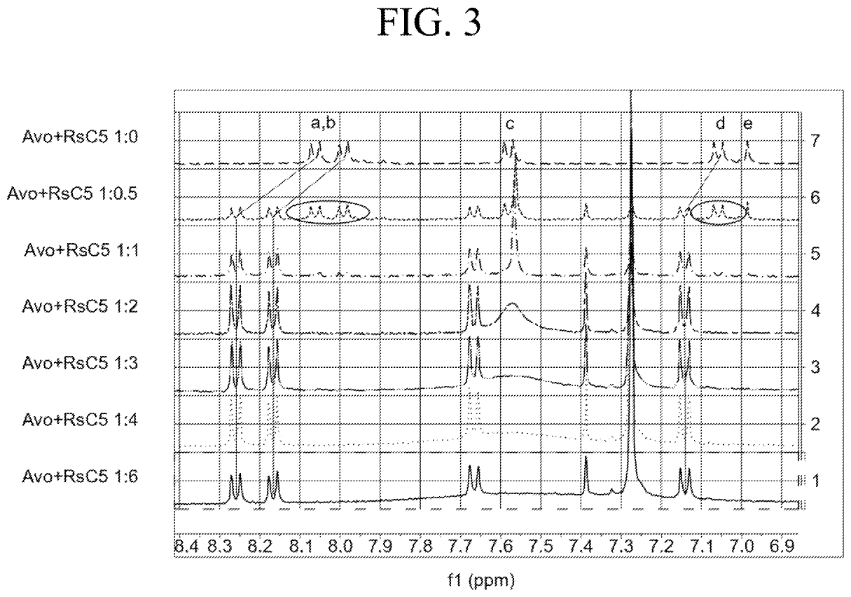

Stock solutions of AVB and RsC5 were prepared using acetonitrile-d3. In different NMR tubes, the host (RsC5) was gradually added to the guest (AVB) at molar ratios from 0:1 to 6:1, ensuring that each tube contained the same amount of deuterated solvent (0.8 mL). The tubes were sonicated and shaken vigorously until the solutions were well mixed, and then NMR spectra were recorded. The NMR titration (FIG. 3) showed significant changes in aromatic protons on AVB. Aromatic protons a, b, and d showed a significant left shift in the presence of RsC5, which indicated the interaction between AVB and RsC5. When the molar ratio was 0.5:1 (RsC5:AVB), the amount of AVB interacting with RsC5 was as much as the free AVB; when the ratio increased to 1:1, there was no free AVB anymore. This indicated that the combining ratio of AVB and RsC5 was 1:1.

### Example 2

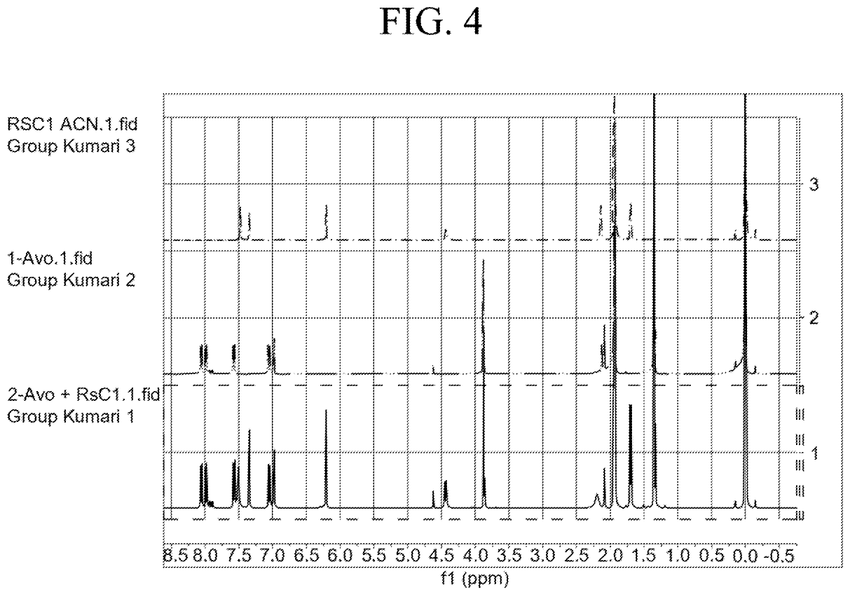

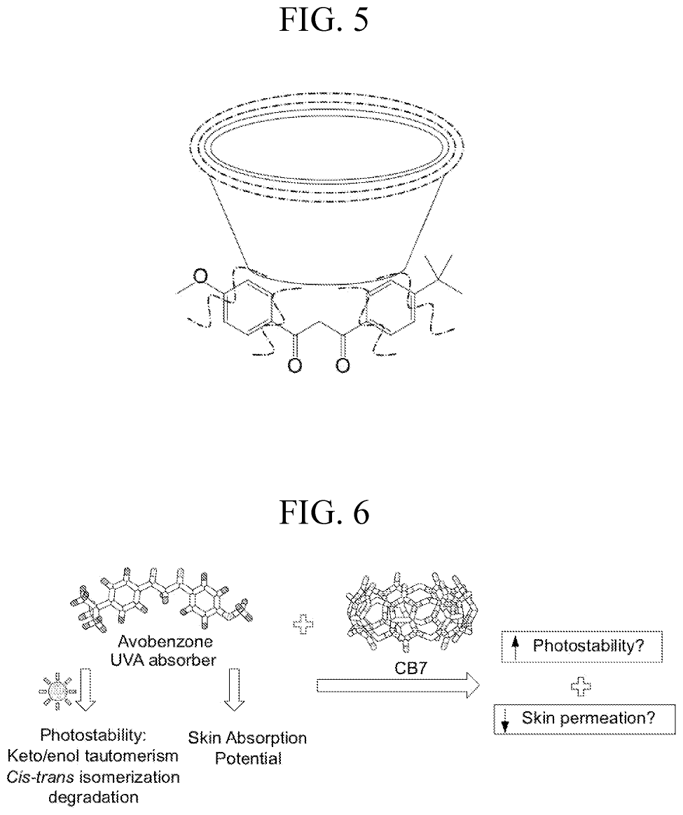

NOESY was taken in acetonitrile-d3 at a ratio of 1:1 (AVB:RsC5). From the spectrum, there were cross-peaks between a, b, and 2, c, and 3 and c and the tail region. Those cross-peaks indicated that AVB was interacting with the lower rim of RsC5. AVB and RsC1 did not make a complex. Maybe the tail length was a significant part of making a successful complex in this situation. Considering all the above information, FIG. 4 predicts the possible confirmation of the complex.

### Example 3

Inclusion complexes of avobenzone and cucurbit[7]uril were prepared by a kneading method. The avobenzone and cucurbituril were ground at room temperature (22±1° C.) using an agate mortar and pestle. Each compound was weighed at varying mole ratios, maintaining a constant total mass of 50 mg. The mole ratios of AVB to CB[7] ranged from 1:0.1 to 1:3. The dried mixture was ground in small quantities of water (˜100 μl each, 3-4 times) to achieve a white homogenous paste. After continuous grinding for 30 min, additional water was added to create a slurry (totaling of about 2-3 mL), which was then transferred to a 20 mL vial. The dried powder of the AVB-CB[7] complex was obtained by freeze-drying.

The dry complexes were characterized by 1H-NMR to confirm the complexation. The photostability profiles of the complexes were collected using UV/Vis spectrophotometer after UV irradiation. The ¹H-NMR results show that cucurbit[7]uril selectively binds to the keto tautomer of avobenzone. Upon UV irradiation, avobenzone complexing cucurbit[7]uril exhibited the least photostability.

### Example 4

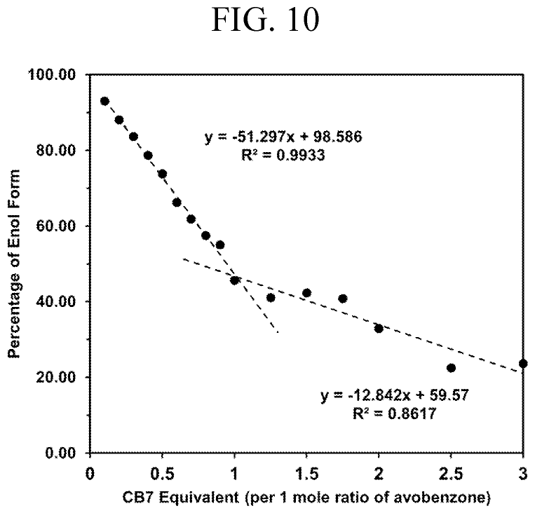

FIG. 10 is a plot of the percentage of enol tautomer as a function of added CB7 equivalent. The percentage of enol tautomer present was calculated by integrating g′- and g-proton resonance peak for all NMR data in DMSO-d6. Percentages of enol tautomer decrease with increasing CB7 to avobenzone molar ratio, indicating CB7 complexes avobenzone by converting free enol tautomer to bound keto tautomer. The 1:1 stoichiometry of avobenzone-CB7 complex can be obtained from intersection point of two linear regression lines.

### Example 5

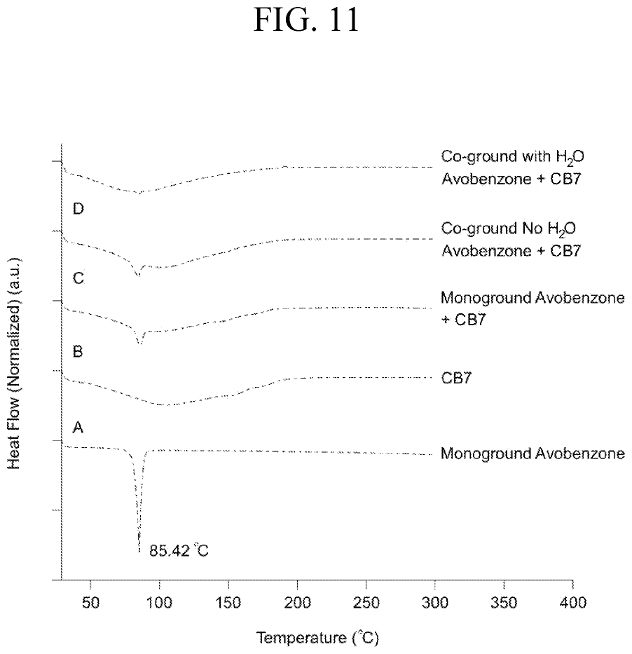

FIG. 11 is a series of DSC curves of: monoground avobenzone (A), CB7 (B), physical mixture of monoground avobenzone and CB7 (1:1 molar ratio) (C), co-ground mixtures of avobenzone and CB7 with no water (1:1 molar ratio) (D), and co-ground mixtures of avobenzone and CB7 with water (1:1 molar ratio) (E). The curve of C and D is the sum CB7 dehydration and the melting peak of avobenzone, indicating no complexation occurs. The curve of E decreases compared to C and D, suggesting an interaction of avobenzone and CB7 and partial complexation. The presence of the avobenzone melting peak indicates that there is still free avobenzone molecule in the mixture. According to NMR data, it suggests that free avobenzone molecule is enol tautomer.

### Example 6

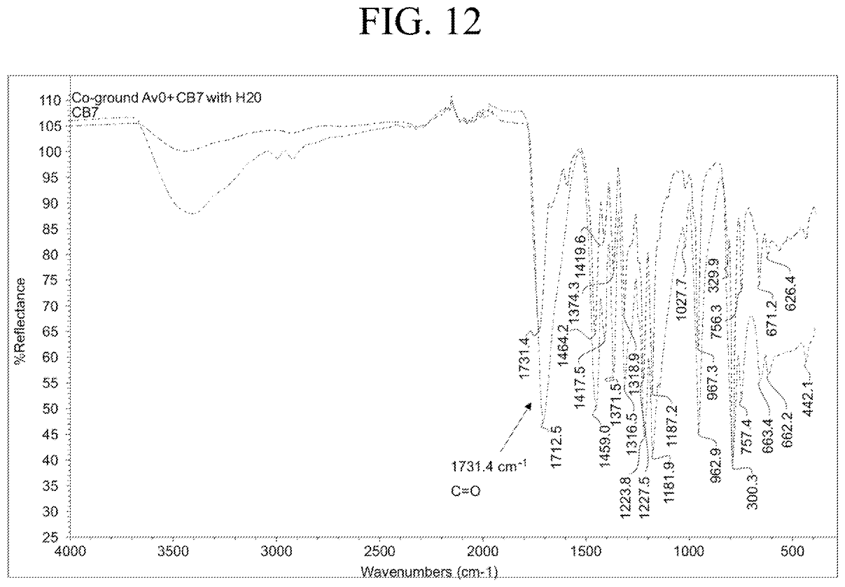

FIG. 12 shows IR spectra of CB7 (green) and mono-ground avobenzone and CB7 (red) (A) and CB7 (green) and co-ground avobenzone and CB7 (red) (B). The C═O brand of CB7 changes the position from 1714 to 1731 cm-1, indicating the interaction at the carbonyl portals of CB7.

### Example 7

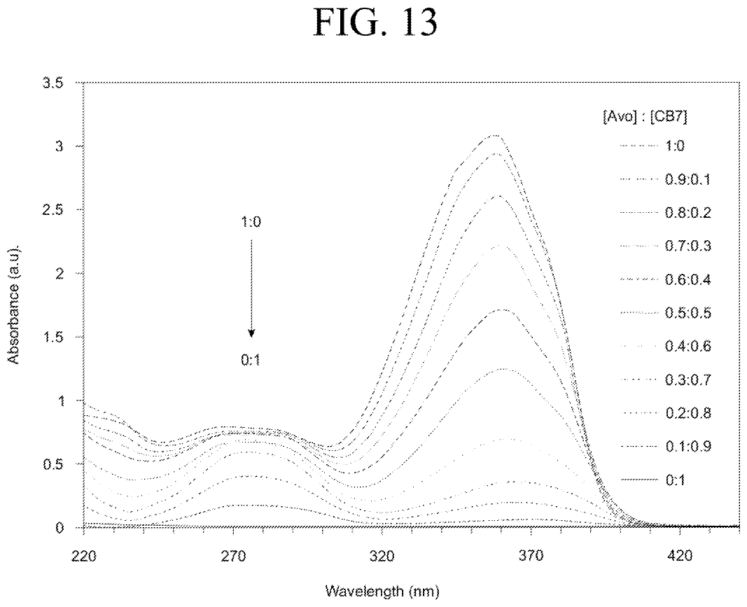

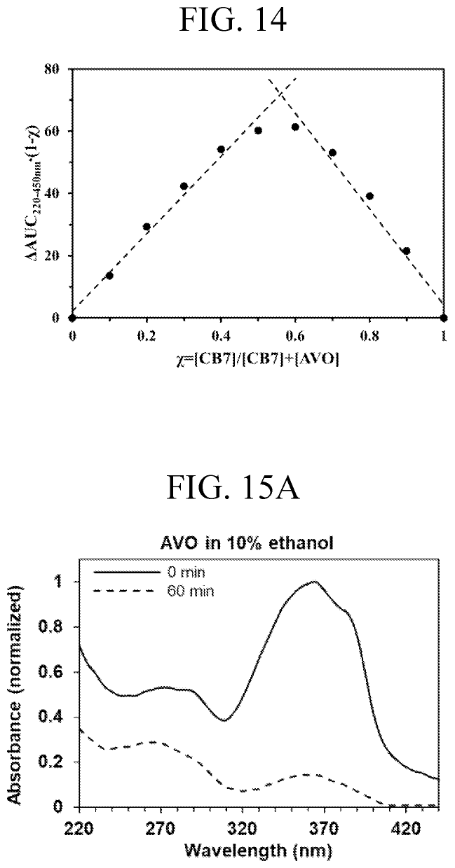

FIG. 13 shows UV-vis spectra of samples prepared for Job's plot. 1×10-4 M stock solutions of avobenzone in methanol and CB7 in water were mixed at different volume ratios. The total volume of the mixtures was kept constant at 4 mL. FIG. 14 shows a Job's plot for avobenzone and CB7 by plotting the difference in Area under Curve (AUC) ranging from 220 to 450 nm versus the molar ratio of CB7. The AUC was used for calculation instead of the maximum absorbance peak at 360 nm due to the equilibrium shift between keto form (absorbance at 270 nm) and enol form (absorbance at 360 nm) of avobenzone. The stoichiometric ratio between avobenzone and CB7 was determined to be 0.43:0.57.

### Example 8

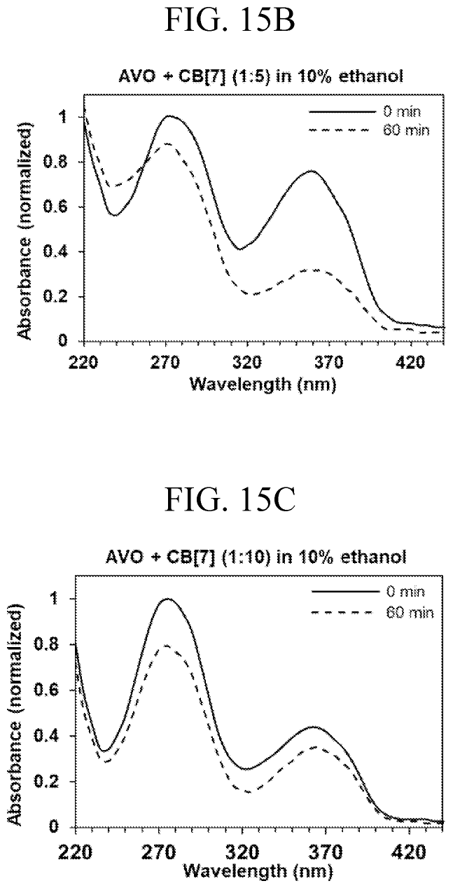

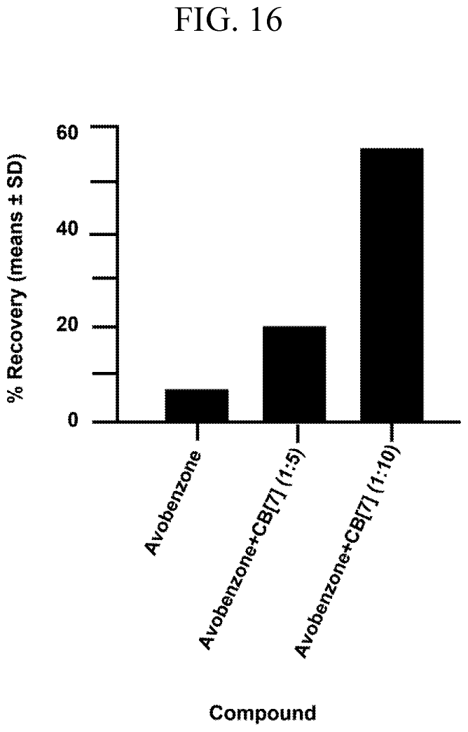

UV spectra of AVB alone in 10% ethanol/water, AVB-CB[7] (1:5 molar ratio) in 10% ethanol/water and AVB-CB[7] (1:10 molar ratio) in 10% ethanol/water before and after 60-min irradiation are overlaid in FIGS. 15A-15C. The results are expressed as % recovery in FIG. 16. The results show improved reduction in absorbance for the AVB-CB[7] complexes.

All documents cited are incorporated herein by reference; the citation of any document is not to be construed as an admission that it is prior art with respect to the present invention.

It is to be further understood that where descriptions of various embodiments use the term “comprising,” and/or “including” those skilled in the art would understand that in some specific instances, an embodiment can be alternatively described using language “consisting essentially of” or “consisting of.”

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

What is claimed is:

1. A composition comprising the complex avobenzone-RsC5.

2. A sunscreen composition comprising the composition of claim 1 and a cosmetically acceptable carrier.

3. The sunscreen composition of claim 2 wherein the complex avobenzone-RsC5 comprises from about 0.5 to about 10 weight percent of the sunscreen composition.

4. The sunscreen composition of claim 2 further comprising one or more film formers.

5. The sunscreen composition of claim 2 further comprising one or more emulsifiers.

6. The sunscreen composition of claim 2 further comprising one or more silicone oils.

7. The sunscreen composition of claim 2 wherein the sunscreen composition is in the form of a lotion, cream, gel, or spray.

8. The sunscreen composition of claim 2, wherein the cosmetically acceptable carrier comprises water.

9. A composition comprising the complex avobenzone-cucurbit[7]uril.

10. A sunscreen composition comprising the composition of claim 9 and a cosmetically acceptable carrier.

11. The sunscreen composition of claim 10 wherein the complex avobenzone-cucurbit[7]uril comprises from about 0.5 to about 10 weight percent of the sunscreen composition.

12. The sunscreen composition of claim 10 further comprising one or more film formers.

13. The sunscreen composition of claim 10 further comprising one or more emulsifiers.

14. The sunscreen composition of claim 10 further comprising one or more silicone oils.

15. The sunscreen composition of claim 10, wherein the sunscreen composition is in the form of a lotion, cream, gel, or spray.

16. The sunscreen composition of claim 10, wherein the cosmetically acceptable carrier comprises water.
